(19) United States
(12) Patent Application Publication
(10) Pub. No.: US 2008/0296154 A1
(43) Pub. Date: Dec. 4, 2008

(54) Analysis Device

(75) Inventors: John Dilleen, Stirling (GB); David Williams, Auckland (NZ)

Correspondence Address: FOLEY HOAG, LLP;PATENT GROUP (w/ISA), 155 SEAPORT BLVD., BOSTON, MA, 02210-2600, US

(73) Assignee: INVERNESS MEDICAL SWITZERLAND GMBH, Zug (CH)

(21) Appl. No.: 11/910,369

(22) PCT Filed: Mar. 20, 2006
(86) PCT No.: PCT/GB06/00993

§ 371 Date: Jul. 14, 2008

(30) Foreign Application Priority Data
Mar. 31, 2005 (GB) 0506598.2

(51) Int. Cl. G01N 33/50

(52) U.S. Cl. 204/403.01

(57) ABSTRACT

An analysis device is provided for analysing a substance sample. The device comprises a plurality of sample activatable battery cells connected in series.

The invention relates to an analysis device in particular for analysing a substance sample.

Simple disposable assays for the detection of analytes are known, for example the ClearBlue Digital™ device sold by Unipath Ltd for the detection of the pregnancy hormone human chorionic gonadotropin. Provision of an electronic assay device represents various advance over traditional visually read devices in that results of a test may be displayed without user interpretation, the result may be semi or totally quantitative and the results may be stored into memory. However such devices may include a number of photodiodes and photodetectors as well as electronics to process the signal and an LED display, all of which have certain power requirements. These devices are typically powered by commercially available button battery cells provided within the device. Environmental regulations in some countries require that the batteries are able to be removed from the device which increases cost. Furthermore there are environmental implications involved with indiscriminate disposal of such devices as well as farther cost implications such as the cost of the battery itself. Therefore alternative sources of power would be desirable.

One known analysis device is described in WO00/33063 and comprises a sensor for electrochemical detection of a sample. Power for the detector is provided by a pair of electrodes of dissimilar material which generate current when sample passes between them. A problem with known arrangements is that only low voltages can be achieved, governed by the materials forming the electrodes.

The invention is set out in the claims. In particular because of the provision of a plurality of sample activateable battery cells connected in series, increased voltages and tailored voltage levels can be obtained. This enables electronic elements having high voltage requirements such as a photodiode or LED display to be included within a device without the need to provide a source of power from a separate battery. By making the battery cells activateable by the fluid sample to be tested, the need to introduce a separate source of electrolyte is removed, making the device simple to use. Furthermore, the sample activateable battery cells may be mass-produced simply, cheaply and conveniently, for example by screen printing a large number of battery cells onto a base substrate and subsequently cutting to provide individual assay or analysis devices.

Embodiments of the invention will now be described, by way of example, with reference to the drawings, of which:

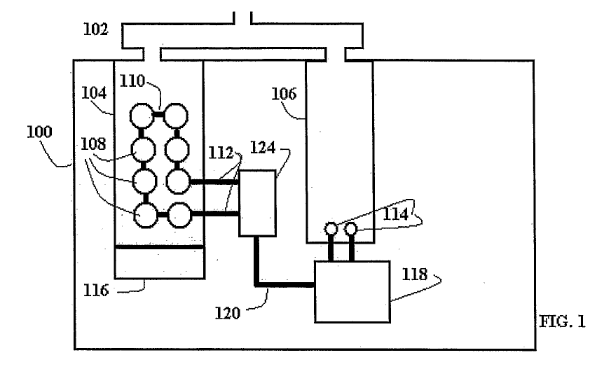

FIG. 1 is a schematic diagram showing components of the invention;

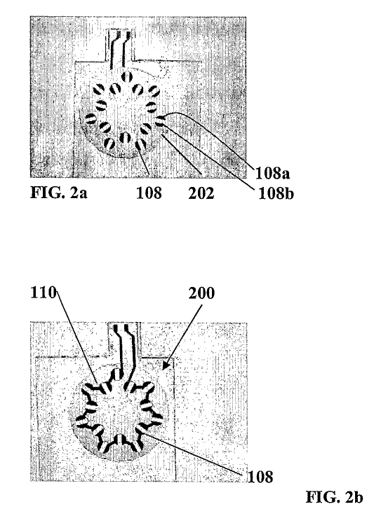

FIG. 2a shows, in plan view, an electrode configuration according to the invention;

FIG. 2b shows, in reverse plan view, the electrode configuration of FIG. 2a;

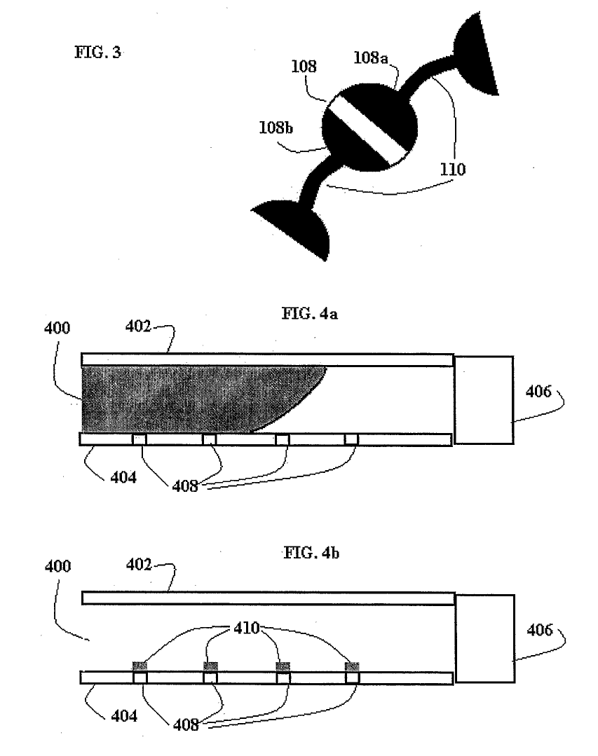

FIG. 3 shows a detail of the electrode configuration according to the present invention;

FIG. 4a is a sectional side view showing wetting of the electrodes during a filling stage;

FIG. 4b is a sectional side view showing wetting of the electrodes subsequent to the filling stage;

FIG. 5 shows an alternative electrode wetting configuration;

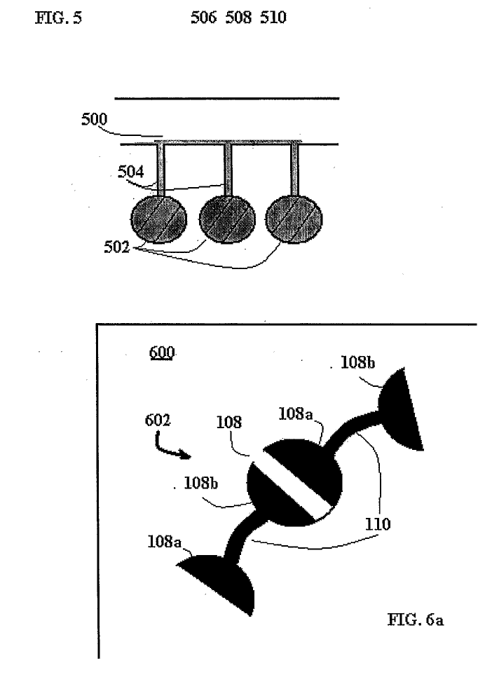

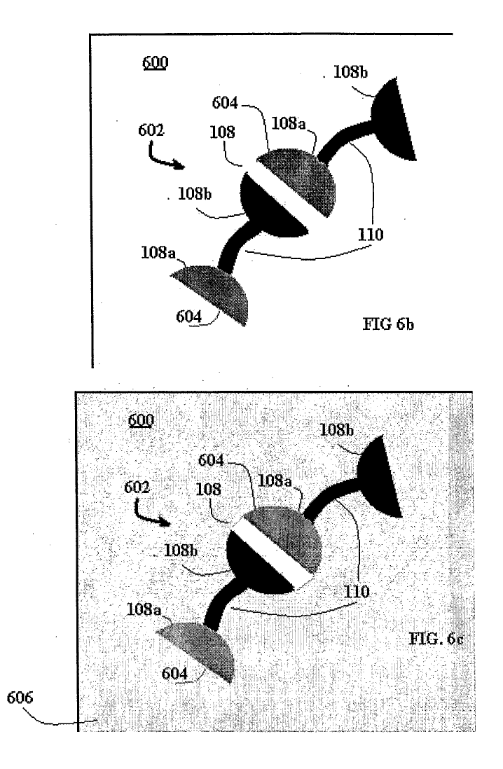

FIG. 6a shows a first fabrication step according to the present invention;

FIG. 6b shows a second fabrication step according to the present invention;

FIG. 6c shows a third fabrication step according to the present invention;

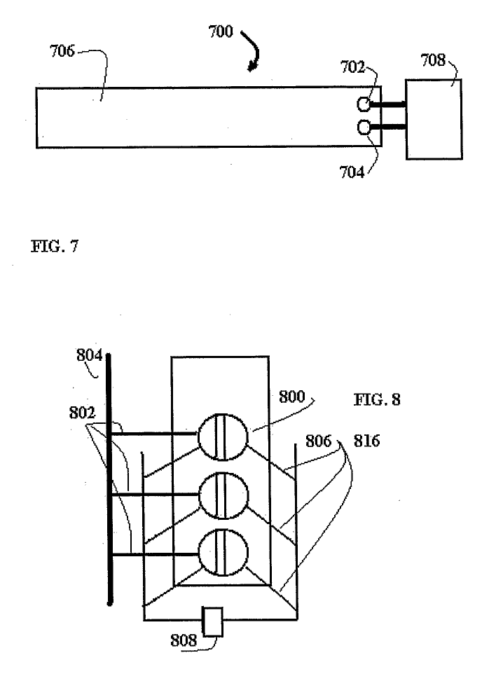

FIG. 7 shows an alternative detector arrangement according to the present invention; and

FIG. 8 shows an alternative electrode configuration according to the present invention.

In overview the invention comprises an assay or other analytical device for example for analysis of bodily fluids such as blood or urine having a plurality of battery cells which are activated by the sample under analysis. The batteries are connected in series such that the voltages sum to provide a higher voltage.

Referring to FIG. 1, the device designated generally 100 can be seen schematically in more detail. Sample is delivered by a sample delivery channel 102 to each of a power channel 104 and a sample analysis channel 106. The power channel 104 includes a plurality of energy cells such as galvanic cells or battery cells 108 each comprising a pair of electrodes activated by the sample to generate current. The cells are connected in series via connections 110 and deliver a current via connections 112. The sample analysis channel 106 includes sample analysis electrodes 114 which can of any appropriate detector or analyser type. The power channel 104 includes a wick 116 to draw sample along the channel—a similar wick may be provided (not shown) on the sample analysis channel 106 as well. The detector electrodes 114 in sample analysis channel 106 are connected to an analysis/indicator device 118 for example comprising circuitry for analysis of the sample and indication of an analysis result. The analysis indicator device 118 is powered by the battery cells 108 in the power channel 104 via connections 112. Optionally a controller 124 is also provided in the circuit providing power and control commands to the sample and analysis unit 118 via a connection 120.

Referring to FIGS. 2a and 2b a specific electrode configuration can be seen in more detail in FIGS. 2a and 2b. In particular the individual battery cells 108 comprise electrode anode-cathode pairs 108a, 108b in facing semi-circular form with a sample channel therebetween and connected on the underside via connections 110. The plurality of sample activatable battery cells 108 are provided in a generally circular configuration purely for packing purposes and in a staggered concentric arrangement whereby adjacent radially external and internal cells are successively connected in series. In the embodiment shown the cells are provided on a substrate 200 which is further overlaid with a barrier layer 202 having a sample delivery face visible in FIG. 2a and an electrical connection face on which the electrical connections are provided as shown in FIG. 2b on the underside. The barrier 202 is formed of hydrophobic or otherwise sample repelling material whereas the battery cells 108 are formed of hydrophilic or otherwise sample attracting material. This ensures that when sample is introduced to the sample delivery base of the arrangement as shown in FIG. 2a it collects only at the cells and does not form short circuits between the cells, electrical connectivity between the cells being provided by the connections on the underside.

It will be seen that, as a result, when sample is provided to the arrangement a plurality of voltages are generated at each battery cell and, as these are connected in series, a significantly larger summed voltage can be achieved. In addition the voltage achieved can be tailored by selecting, during design, the number of cells used and, as appropriate, the specific connectivity configuration. Yet further tolerance and redundancy can be built into the arrangement such that short circuiting of some cells will not reduce the voltage below a predetermined threshold.

Referring now to FIG. 3 a detail of a battery cell can be seen viewed from the underside of the arrangement. The cell 108 is connected to adjacent cells via connection 110 and includes spaced electrodes 108a, 108b. One of the electrodes acts as an anode and the other as a cathode when sample is introduced such that the electrodes operate as batteries. In particular, as discussed in more detail below, one of the electrodes 108a, 108b can be coated in an appropriate material to provide a potential difference upon addition of sample, for example, in the form of a sample-soluble reagent coating.

In operation, sample is delivered to the cell arrangement for example by capillary action and settles on the hydrophilic electrode portions acting as a reagent in conjunction with the electrode material or coatings and also as an electrolyte between the electrodes as a result of which a voltage is generated. Because a plurality of samples are provided in series the voltages sum to give the desired output voltage. In the case of short circuits between adjacent cells, the circuit is not broken and the voltage is reduced by a comparatively small amount for a large number of cells whilst keeping the same current. One possible usage of the cells is in one-use or limited period-use devices where depletion of the materials in the cells is over a long enough timescale so as to be insignificant. In particular, surprisingly, even though no membrane is provided between the electrodes in the cell, there is little or no significant poisoning of the electrodes by transport of the sample across the space between the electrodes as a result of the constricted timescale or number of uses. Accordingly a simplified construction is obtainable.

FIGS. 4a and 4b show one possible delivery model for ensuring that the sample reaches the cells. In particular the power channel designated generally 400 comprises an upper wall 402 and a lower wall 404 spaced sufficiently to allow capillary action to draw sample along the channel 400. In addition a wick or any other component with a strong capillary pull can be provided at a distal end of the channel 400 to ensure flow of the sample. A plurality of electrodes 408 are provided in the lower wall 404 of the type described above and electrically connected on the underside of the wall 404, that is, the opposite side to that along which the sample is drawn. In order to draw sample along the channel the upper wall is hydrophilic. As a result although the barrier component of the lower wall 404 separating the electrodes 408 is, as discussed above, hydrophobic, sample is drawn across the upper, sample delivery surface allowing it make contact with and settle on the hydrophilic battery cells 408. In particular, as can be seen, the sample proceeds along the channel 400 with a “leading edge” at the upper hydrophilic wall base 402. As a result, as shown in FIG. 4b, when the sample has passed, some rests on the battery cells 408 as shown at 410 allowing activation of the batteries and creation of a voltage to power the analysis device as described above.

An alternative arrangement is shown in FIG. 5 in which a delivery channel 500 is connected with a plurality of cells 502 via respective capillary activation channels 504. As can be seen, sample rests in the activation channels 504 and on the electrodes 502 but, after sample has passed through the channel 500, is otherwise evacuated. Even if a small amount of sample remains on a surface of the delivery channel in the vicinity of activation channel 504 it will be seen that the layer will be very thin such that its resistivity is high enough not to conduct between and short circuit the cells.

Referring now to FIGS. 6a to 6c, the fabrication steps involved in constructing the cell assembly can be better understood. As seen in FIG. 6a, the cell configuration 600 is screen printed onto a substrate 602. The screen printing can be done by any appropriate means and followed by a drying stage as appropriate. In particular the substrate can comprise a polycarbonate sheet and the cell configuration printed in a “dumbbell” configuration whereby respective halves of each adjacent cell and the connection therebetween are printed alongside one another in any desired configuration. The screen printable carbon paste itself may be any appropriate material for example known by code C2000802D2 (Gwent Electronic Materials Limited, Wales).

In order to provide dissimilar materials at opposing electrodes in a cell, in the next step shown in FIG. 6b a zinc powder doped carbon paste of the type described above with a 45% zinc loading of zinc dust, for example of particle size less than 10 microns and 98+% Sigma-Aldrich batch 11010EA [is printed onto one of each electrode pair, for example electrode 108a to provide an anode 604. The second of each electrode pair may comprise the undoped carbon paste to provide a cathode. Alternatively, carbon paste doped with copper powder or any other suitable metal powder may be printed onto the second of each electrode pair to provide a cathode.

Referring now to FIG. 6c a dielectric layer 606 for example in the form of dielectric paste D60202D1 is then printed onto the substrate 600 being masked in the vicinity of the cells 108 but overlaying the connections 110 to provide a barrier layer between the sample delivery face and electrical connection face, the dielectric layer providing the required hydrophobic properties.

Finally a sample delivery channel is defined by providing a spaced upper layer having a hydrophilic inner face, of the type described above, and, as appropriate, a wick to ensure sample flow across the face. As a result sample settles only on the cells where it provides an electrochemical reaction and electrolyte properties to activate the cells and provide a voltage. Dependent on the sample it may be desired to enhance its electrolytic properties for which purposes, for example, a surface of the channel may be coated with a sample-soluble conductive material such as a salt which is dissolved in the sample to provide the desired electrical properties. Optionally, the surface may be coated with a salt such as copper sulphate acidified with potassium hydrogen sulphate in a suitable polyvinyl alcohol (PVA) or other polymer solution, such as Sigma-Aldrich 85k to 146k molecular weight PVA. Increasing the PVA concentration in the salt/PVA solution increases its viscosity, which is useful for screen printing. In addition, dissolving the salts in PVA allows slow release of the salts so that they are not washed away by the sample on wetting, and allows an even and homogenous layer of salts to be deposited. The hydrophilic nature of this layer is due mainly to the salt content and is increased by the PVA also retaining moisture.

In their simplest form each cell comprises a zinc (in carbon paste) anode and a carbon cathode in an acidified salt solution such as copper sulphate acidified with potassium hydrogen sulphate. When the sample activates the cell the zinc is oxidised and the copper ions (from the salt) are reduced at the anode. In an opposing reaction at the cathode, H+ ions are converted to hydrogen gas. The theoretical voltage of each cell is 1.1V when measured with a high impedance voltmeter, however it has been found in practice that the chemistry described above for each cell generates approximately 0.5V meaning that the device as a whole generates a voltage of 7V.

It will be seen that any cell configuration can be adopted including the concentric circular configuration shown in FIGS. 2a, 2b or a linear configuration. Looped configurations are shown as these reduce the length of the device and also ensure that sample only has to pass a reduced distance to activate the cells; any alternative device including more tortuous paths can of course be accommodated. Similarly any number of cells can be adopted. In addition the current provided by the circuit can be tailored appropriately for example by varying the size or surface area of the cells appropriately.

In an alternative fabrication method, the completion or perturbation of a battery may occur due to an immuno recognition event. The use in standard lateral flow immunoassays of metal solutions, for example 80 nm metal particles of silver or gold, with antibody immobilised is well known in the art. A carbon electrode such as those described above may be coated with antibody and the metal delivered to it in the presence of antigen analyte. This forms a sandwich, resulting in a carbon electrode with a metal particle coating which could form one electrode of the cell, hence completing the battery and getting current to flow. Alternatively, this method could be used to poison an otherwise satisfactory system to lower its current and/or voltage. A measurement relating to analyte concentration can be obtained from the system based on the resulting electrical characteristics for example current or voltage level.

It will be seen that alternative configurations and circuitry options can be adopted depending on the desired properties of the device. For example the cells can power, via a controller, a selectively switchable time gate to one or more sample analysis channels such that different analysis channels can be opened at different times which may be of benefit in complex or time-varying analysis methodologies, for example involving multiple analysis steps. In that case any appropriate time gate can be used, for example an electro-mechanical time gate or, for example, a graphite barrier coated on or around the channel. As is well known graphite is hydrophobic in normal configuration but, when a suitable charge is applied, it may become hydrophilic due to oxidation of the graphite surface to form graphite oxide. Thus the cell in conjunction with control circuitry could apply a charge to such a barrier at a predetermined time to change the surface tension properties of the barrier thus controlling the flow of sample fluid across it.

In an alternative detector configuration shown in FIG. 7 a detector 700 comprising electrodes 702, 704 in a channel 706 provides a signal to an indicator or analyser unit 708 when sample passes through the channel and creates a circuit between the electrode 702, 704. In addition if the electrode 702, 704 are formed of differing materials, for example coated as described in more detail above in an anode-cathode configuration then when sample reaches the detector it can power the detector such that the electrodes serve both as detection and power components for the analysis and indicator unit 708.

Referring to FIG. 8 it will be seen that a further alternative power cell configuration is shown. In particular a plurality of cells 800 are provided including individual activation channels for delivery of fluid, 802, fed by a common delivery channel 804. The electrodes are electrically connected in parallel via connections 806 to provide power to an appropriate component 808 which may be for example an analysis or indicator unit as described above. In addition the activation channels 802 are individually switchable between open and closed configurations for example using a time gate of the type described above under the control of any appropriate controller. As a result the number of cells activated in the circuit in parallel at any time can be varied as appropriate. This ensures that whilst a constant voltage is provided, the current can be varied in any desired manner to provide a controllable and tailorable current delivery to the device. It will further be seen that instead of individual cells provided in parallel, sets of cells may be provided in parallel wherein each individual set is connected internally in series increasing the voltage accordingly. It will further be seen that, using activatable channels of the type described, more complex power control operations can be introduced such as time delayed switching of cells, for example where a first cell is expected to decay prior to completion of analysis.

As a result of the arrangement described a self-powered high voltage analysis device is provided allowing additional control and tailoring of electrical characteristics and fabricated in a simple manner with a minimal number of components. In particular no membrane is required in between the electrodes for single use or short-term use devices and yet further, only one metal (zinc) is required, the uncoated carbon electrode operating in conjunction with it, although appropriate metal coatings can be applied to both electrodes as appropriate.

It will be appreciated that the various components and materials described above can be varied as appropriate as long as the desired effects are attained. Any type of cell can be adopted for example using soluble reagents or different metals for each electrode and any appropriate configuration of cells can be adopted. Similarly the detector itself can be of any appropriate type as will be well known to the skilled reader. The sample for analysis can be a bodily sample or can be an industrial sample for example sea or river water or industrial pollutants. In the case that use of hydrophobic/hydrophilic coatings provides the desired sample flow then any aqueous sample can be used, if necessary in conjunction with soluble conductive coatings to provide the desired electrical properties, or generally it will be appreciated that any flowable sample can be used in the appropriate configuration. Similarly the cells can be used in any sample analysis or detection devices having integral electronic components such as photodiodes, photodetectors, display and memory means and computation means. For example, the device described herein has been shown to power a light-emitting diode (LED) for approximately 40 minutes. The power source could additionally be used to power other elements such as electromechanical time gates, chemical time gates, fluid pumping means such as electroosmotic pumping and so on. The device may be chosen from for example an assay or analytical device, a sample delivery or manipulation device, a device to remove fluid sample from a source such as removal of interstitial fluid from a subject, and so on. The device may be disposable. The power requiring elements of the device may be incorporated as part of the device itself or may be connectable to the device. The device may be for example a lateral flow assay device or a microfluidic device. The device may comprise one or more separate sample activateable power sources which may have the same or differing power characteristics. The electrolyte for the power source may be derived from the fluid sample under analysis or may be provided separately in addition to the fluid sample.

1. An analysis device for analysing a substance sample, the device comprising a plurality of sample activatable battery cells connected in series.

2. A device as claimed in claim 1 in which the cells comprise sample attractive regions and respective cells are separated by sample repelling regions.

3. A device as claimed in claim 2 in which the sample is an aqueous sample, the sample attractive regions are hydrophilic regions and the sample repelling regions are hydrophobic regions.

4. A device as claimed in claim 1 further comprising a sample delivery channel.

5. A device as claimed in claim 4 in which the sample delivery channel comprises a capillary action sample delivery channel.

6. A device as claimed in claim 4 in which the cells are provided on a channel surface.

7. A device as claimed in claim 6 in which the channel further comprises a sample attractive surface opposing the surface on which the cells are provided.

8. A device as claimed in claim 4 further comprising a respective activation channel for delivering sample from the sample delivery channel to each cell.

9. A device as claimed in claim 4 further comprising a wick associated with the sample delivery channel to draw sample along the channel.

10. A device as claimed in claim 4 in which a portion of the sample delivery channel bears a soluble conductive material.

11. A device as claimed in claim 1 in which the cell comprises an electro-chemical cell.

12. A device as claimed in claim 11 in which the electro-chemical cell is activatable by a fluid sample acting as an electrolyte.

13. A device as claimed in claim 12 in which the cell further comprises a reagent which serves to increase the ionic conductivity of the fluid sample.

14. A device as claimed in claim 13 in which the reagent is a salt.

15. A device as claimed in claim 1 in which each cell comprises a pair of spaced electrodes defining an uninterrupted flow space therebetween.

16. A device as claimed in claim 1 in which the battery cells are arranged so as to minimise connection length therebetween.

17. A device as claimed in claim 16 in which the cells are provided in a staggered configuration.

18. A device as claimed in claim 16 in which the cells are provided in a concentric configuration.

19. A device as claimed in claim 1 in which the cells extend through a sample barrier having a sample delivery face and an electrical connection face.

20. A device as claimed in claim 1 in which the cell also acts as a sample detector.

21. A device as claimed in claim 1 further comprising a sample analysis channel communicating with a sample analysis component.

22. A device as claimed in claim 21 in which the sample analysis channel is selectively switchable between an open and closed configuration.

23. A device as claimed in claim 1 further comprising a controller unit for controlling sample delivery.

24. A device as claimed in claim 1 in which the sample activatable cell is arranged to form a cathode or anode material upon contact with sample or sample carrier fluid.

25. A device as claimed in claim 24 in which the cell has an electrode coating arranged to attract anode or cathode material from sample carrier fluid upon contact with sample.

26-40. (canceled)
